(19) United States
(12) Patent Application Publication
(10) Pub. No.: US 2016/0022482 A1
(43) Pub. Date: Jan. 28, 2016

(54) Cooling Products and Methods

(71) Applicant: Gawi Healthcare, LLC, Paterson, NJ (US)

(72) Inventors: Carol M. Forden, Lakewood Ranch, FL (US); Laurie A. Forden, Lakewood Ranch, FL (US)

(73) Assignee: GAWI HEALTHCARE, LLC, Paterson, NJ (US)

(21) Appl. No.: 14/878,516

(22) Filed: Oct. 8, 2015

Related U.S. Application Data
(62) Division of application No. 12/852,042, filed on Aug. 6, 2010.
(60) Provisional application No. 61/232,565, filed on Aug. 10, 2009.
(60) Provisional application No. 61/232,564, filed on Aug. 10, 2009.
(60) Provisional application No. 61/232,677, filed on Aug. 10, 2009.

(51) Int. Cl. A61F 7/10; D06M 13/148; D06M 13/207; D06M 15/333; D06M 15/356

(52) U.S. Cl. CPC A61F 7/10; D06M 15/333; D06M 15/3562; D06M 13/207; D06M 13/148; A61F 2007/0098

(57) ABSTRACT

Cooling products and methods of manufacture are disclosed. The cooling product is comprised of a substrate impregnated with a polymer gel and an antimicrobial agent. The product may be a compression wrap, pad, body wrap, container wrap, blanket or article of clothing.

### RELATED APPLICATIONS

This application is a divisional of, and claims the benefit of priority to, U.S. patent application Ser. No. 12/852,042 filed on Aug. 6, 2010, which is a non-provisional of, and claims priority to, U.S. Patent Application No. 61/232,565, filed on Aug. 10, 2009, U.S. Patent Application No. 61/232,564, filed Aug. 10, 2009, and U.S. Patent Application No. 61/232,677, filed on Aug. 10, 2009, the entire disclosures of which are expressly incorporated herein by reference.

### BACKGROUND OF THE INVENTION

1. Field of the Invention

Cryotherapy is defined as the therapeutic application of a substance or product to the body that removes heat from the body, resulting in decreased tissue temperature.

Compression is often used in conjunction with cryotherapy. Benefits of compression may include improved contact between the skin and the cold source, greater reduction of blood flow to the region, and an increased insulation effect, which may further reduce tissue temperatures. Compression also may assist with control of edema formation that may arise after injury or secondary to microtrauma sustained during a hard workout.

Cryotherapy, or cold therapy, may diminish pain, metabolism, and muscle spasm, thus minimizing the inflammatory response and improving recovery after soft tissue trauma.

2. Related Art

Various methods such as ice packs, ice towels, ice massage, gel packs, cooling sprays, cold immersion, refrigerant gasses and inflatable splints are available for implementing cryotherapy. Single-use chemical-reaction pouches and reusable cryogel packs are also used for cryotherapy. The single-use pouch is a flexible plastic bag that contains two chemicals that come into contact only when the bag is squeezed. This action ruptures an inner plastic membrane, allowing the two chemicals to mix, creating an endothermic reaction. The resulting cold is not sustained and the product must be discarded after its single use. Because of their potentially extreme temperatures (as low as minus 20 degrees Centigrade), frozen cryogel-filled or gel-packs should be used with extreme caution and the user should always place a towel between the gel and the skin to limit potential frostbite injury to the skin, nerve damage, and increased swelling and inflammation. Open-cell foam gel cold wraps are also used but have several limitations including consumer distaste for the small sandy granules and large chunks of polyvinyl alcohol (PVA) and PVA/gel particles that fall off the product upon opening and throughout wearing this product.

Accordingly, a need exists for a cooling product that reduces body heat without subjecting a user to potentially extreme temperatures, maintains its integrity over time, and is preferably reusable.

There is also a need for a cooling product that reduces or maintains the temperature of items such as food and beverage storage containers, medicines, medical-related substances such as pharmaceuticals such as insulin and other biologicals, tissue samples and blood, that eliminates or reduces the need to transport ice or ice water, and does not require an outside power source or cleanup due to condensation, melting, or spills.

### SUMMARY

Illustrative embodiments of the invention include cooling products that may be used for cryotherapy and cryotherapy with compression used in the treatment of a variety of diseases and ailments and may draw the heat out of covered tissues, aid in mending injuries and speed postoperative healing. Products embodying the disclosed technology may offer the user the ability to apply such cryotherapy with little advance preparation as is required for other solutions, and often with lower weight. The ideal application for such inventions includes, but is not limited to, individuals engaged in firefighting, sports, and strenuous work activity in a hot environment, as, for example, in the case of military personnel in both training and combat situations.

The disclosed cooling products may also be used to reduce or maintain the temperature of bottles and other containers that may contain food and medicines.

The cooling products generally comprise a substrate impregnated with a polymer gel. An antimicrobial agent can be incorporated into the product. The gel may have a PVA, PVP, or PVA/PVA blend base for example.

The substrates may be woven, nonwoven, or knit. In an illustrative embodiment of the invention the cooling product is a compression wrap wherein the substrate is a knit, elastomeric polymer/cotton blend wherein the percent elongation of the substrate is greater in the length than in the width. In a further illustrative embodiment of the invention, the cooling product is a pad wherein the substrate is a needlepunched nonwoven polyester or polyester/rayon blend.

The cooling product may be formed into other items such as body wraps, container wraps, articles of clothing such as vests and gauntlets, pads and blankets, for example.

The cooling product may have a release liner on one or both sides, and a protective covering on one or both sides.

The invention further includes methods for manufacturing the cooling products. In an illustrative embodiment of the invention, the method includes subjecting a porous substrate to a first aqueous solution comprised of one or more polymers, an antimicrobial agent, a gel creating or enhancing agent, and the balance substantially water. The wetted substrate is then subjected to a second aqueous solution comprised of an inorganic coagulating agent, an antimicrobial agent; a plasticizer, and the balance substantially water. The impregnated substrate is then sufficiently dried, and formed, such as by die cutting into the desired product.

### BRIEF DESCRIPTION OF THE DRAWINGS

An illustrative manufacturing process and resulting illustrative products can be understood with reference to the following figures:

FIG. 1 depicts an immersion process according to an illustrative embodiment of the invention.

FIG. 2 is a perspective view of a cryotherapy cooling wrap according to an illustrative embodiment of the invention.

FIG. 3 is an enlarged fragmentary sectional view taken substantially along the line II-II in

FIG. 2 according to an illustrative embodiment of the invention.

FIG. 4 illustrates one application of the cryotherapy cooling wrap according to an illustrative embodiment of the invention.

FIG. 5 illustrates another cryotherapy cooling wrap according to an illustrative embodiment of the invention.

FIG. 6 is a perspective view of a cryotherapy vest according to an illustrative embodiment of the invention.

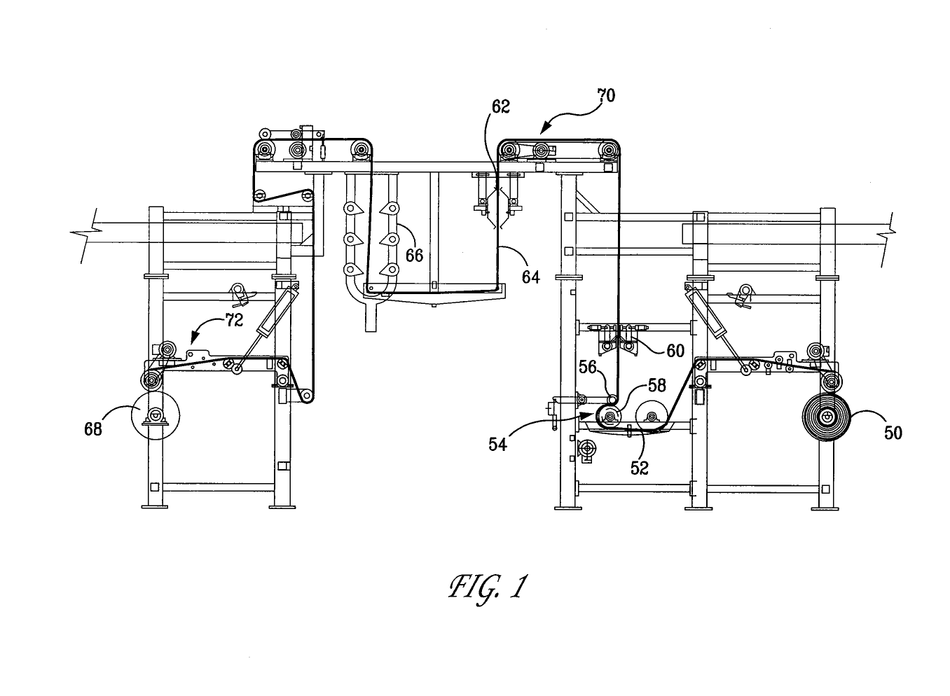

FIG. 7 is an enlarged fragmentary sectional view taken substantially along the line II-II in FIG. 1 according to an illustrative embodiment of the invention.

FIG. 8 is an enlarged fragmentary perspective view illustrating the thin fabric adhered to the substrate to enhance water wicking from the skin surface according to an illustrative embodiment of the invention.

FIG. 9 illustrates the die cut vest garment with Velcro fasteners according to an illustrative embodiment of the invention.

FIG. 10 illustrates another cryotherapy garment according to an illustrative embodiment of the invention.

FIG. 11 illustrates a container wrap product according to an illustrative embodiment of the invention.

FIG. 12 shows a gauntlet according to an illustrative embodiment of the invention.

FIG. 13 depicts a sleeve wrap according to a further illustrative embodiment of the invention.

FIG. 14 depicts a gauntlet according to a further illustrative embodiment of the invention.

FIG. 15 depicts a blanket according to a further illustrative embodiment of the invention.

FIG. 16 depicts a pad according to an illustrative embodiment of the invention.

FIG. 17 shows the layers of a pad according to an illustrative embodiment of the invention.

### DETAILED DESCRIPTION OF THE INVENTION

Illustrative embodiments of the invention include a water-based, gel-embedded substrate that may be used for example, to ease swelling and pain due to sprains, bruises, insect bites, muscle cramps, headaches, degenerative joint disease, surgery, tendonitis, carpal tunnel, burn care, sports injuries, arthritis, edema, drug-induced peripheral edema, bone bruises, heat stress/stroke, and carpal tunnel.

Exemplary embodiments of the invention may have one or more of the following characteristics: non-toxic, latex-free, biodegradable, “green” and safe for the environment, able to maintain effective cooling of the affected area over an extended period of time, able to conform to the skin thereby maximizing the ability to draw the heat energy from the skin, can be refrigerated for enhanced cooling, and can draw heat out of covered areas.

Embodiments of the invention include a cooling product, generally comprising a substrate impregnated with a polymer gel (also called a “gelling polymer”) and having an antimicrobial agent. The gel may be, for example, a polyvinyl alcohol (PVA)-based gel, a PVA/polyvinylpyrrolidone (PVP) blend-based gel, or a PVP-based gel.

The cooling product is particularly suitable for use as a wrap for treatment of various injuries. An exemplary wrap has dimensions in the range of:

width: about 1 to about 8 inches;

length: about 48 to about 72; and

thickness: about 1/32 inch to about ½ inch.

For added therapeutic benefit, the product can be formed to serve as a compression wrap. The ability of the product to impart compression depends in substantial part on the substrate material used. A knit, Spandex® cotton blend can be used as a substrate if compression is desirable. In a particular embodiment of the invention the percent range of Spandex® in the substrate is from about 1% to about 6%. It is noted that the Spandex® can be blended with materials other than cotton, including synthetic, natural, and synthetic/natural material blends. The cooling product substrate may be woven or non-woven, but should be porous to hold the gel. In an illustrative embodiment of the invention, the substrate is water insoluble, i.e., the substrate does not dissolve in or readily break apart upon immersion in, or contact with, water. The invention includes embodiments in which only a portion or portions of the substrate are impregnated with gel, and also wherein the entire substrate is impregnated with the gel.

Further examples of substrate materials include, open cell polyurethane hydrophilic foam, fine or medium double cell with a minimum density of 2.7 to 3.7 lbs/ft³ and tensile strength ranging from 33.2 to 40.5 psi with water holding capacity of 14 to 22 g/g sponge, polyester, cotton/polyester blends, cotton, cotton/Spandex® blend, rayon/polyester/Spandex® blends, cotton/jersey Spandex®, elastic stretch bandage materials, nylon/Spandex® blends, Spandex®/polyester Lycra® blends, Spandex®/nylon/polyester/cotton blends, cotton/Lycra® blends, nylon/cotton/polyester Lycra® blends, blends of other materials with Lycra® and/or Spandex®, nylon/Spandex® lace blends, polyamide, polyamide blends, non-woven polyester with a stretch enhancer added, high-performance polyamide yarns and polymers, high-performance polyamide yarns and polymers blends, Enkaion®, Enka® yarn, Enka® yarn blends, Verona™—All Purpose Permeable Fabric, polyester filament/filament twill, and any materials that have good permeability qualities and that will serve as the carrier for the gel. In an exemplary embodiment of the invention, the substrate fabric is comprised of a knit, ribbed cotton/Spandex® fabric having a rating (pretreatment dry weight) in the range of 3 to 12 ounces per square yard. A non-woven polyester fabric having a rating (pretreatment dry weight) in the range of 2 to 20 ounces per square yard, and preferably in the range of 3-8 ounces per square yard can also be used for some applications. It is noted that where Spandex® or Lycra® is listed, they may be substituted for other similar or identical elastic polymer materials such as those falling in the category of elastane or elastomeric polymers.

In an illustrative embodiment of the invention, the substrate is made from 30/1 combed cotton and 55 denier Spandex®, circular knit (weft knit), wherein the cotton yarn is plated over the Spandex® and knit into a jersey construction, preferably on a fine cut single knit machine with 28 needles per inch in a 30″ diameter cylinder. The fabric preferable is designed to achieve high shrinkage in the bleaching process in both the length (warp) and width (weft). This shrinkage enables the fabric to be finished with minimum width shrinkage, stretch and curl, but with maximum length stretch to facilitate the wrapping process of the final product. Further in this illustrative embodiment, the greigh fabric is bleached optic white in Jet Dye machines. The bleached fabric is then slit open width and stentered (heat set). An antimicrobial may be also added at this point. The finished optic white fabric is then pigment printed on a rotary screen print machine with, for example, a logo. The printed fabric is then stentered to the final specification of preferably 60″ usable width, at 8.2 oz/square yard (280 gm/square meter).

General illustrative ranges of substrate weight and thickness are: weight—about 1.0 oz/yd² to about 7.5 oz/yd²; thickness about 0.015 inches to about 0.100 inches.

Advantageously, it has been found that impregnating the material prior to treatment with additives such as fire retardants, softeners, etc. greatly improves the adherence of the gel to the substrate. This allows the threads or fibers to be properly coated with the gel. Although most additives pose a problem with adherence, it has been found that good gel adherence can be obtained even if the substrate has been treated with an antimicrobial agent prior to impregnation.

The cooling product may contain a polymer release liner disposed on the impregnated substrate. The liner is put in place after formation of the product and is removed for use. The liner may be formed for example, of polyethylene, polyethylene terphthalate (PET), or other similar non-adhesive material or may consist of knit, woven or non-woven materials. A second liner or covering may be disposed on at least a portion of the cooling product. This liner may also be of various materials, such as knit, woven or non-woven materials. By sandwiching the impregnated substrate between two particular types of liners, the product may breathe without having the product touch the user's skin. The impregnated substrate may be laminated to the liner, such as by pinch rolling for example, or merely pressed in place.

The cooling product may also include a covering over at least a portion of one side of the impregnated substrate that remains on the product when the product is in use. The covering can be made of a material that enhances evaporation of water from the skin. Materials that allow vapor to pass in one direction without allowing fluids to pass in the opposite direction are suitable. Various fabrics and plastic films can be used.

A cooling effect enhancement ingredient, anesthetic, ultraviolet inhibitor, flame retardant, binders, antistatic ingredients, or any combination thereof, may be utilized that are either added to the product after the substrate is impregnated, or incorporated into one of the aqueous solutions used to make the product. The cooling effect enhancement ingredient may be for example, menthol or a menthol containing mixture. The anesthetic ingredient may be for example, lidocaine or a lidocaine containing mixture.

For some applications it is preferable for the product to be water resistant.

The cooling product may include printing or other form of decoration or information on the substrate, such that it is visible even when the substrate is impregnated with the gel.

The cooling product may be sold with a container or bag to accommodate the product and retain and/or replenish moisture.

The cooling product may be sterilized and decontaminated by gamma irradiation.

The methods for using products described herein include enhancing the cooling capability of the products and steps to reuse the product. Due to the thermal cooling properties of the gelled substrate, the cooling product will effectively cool the covered area even when the cooling product is in a room temperature environment. For enhanced cooling, the product can be placed in a refrigerator prior to wearing. The cooling product can be dipped in cold water or ice water, for example for 2-10 seconds, at which time thermodynamic cooling properties of the finished product allow the wrap to conduct the temperature of cold water and/or ice water. (Generally, the product will acquire the temperature of the cold water or iced water and typically hold this temperature for a period of about 30 minutes or more.) For reuse, the products can be rehydrated, for example with a small amount of cold water, sealed in a preferably substantially airtight container and placed in the refrigerator or kept at room temperature until the next use. Or after wearing a cooling product for several hours it can be reversed so the part against the users body is now on the outside (in the case of a wrap it can be rerolled from the outside in so that the far outside layer is now the most inside layer); and then placed under water (preferably cold water). Once the product is removed from the stream of water it can be squeezed to remove excess water. The cooling product can then be returned to the resealable pouch or container. The pouch or container can be placed in the refrigerator or in a cabinet until it is needed again. Subsequent applications (6 to 8 times) have been demonstrated to be effective for several hour durations. In an exemplary embodiment of the invention, the product is packaged in a reusable container or is sold with such a container.

Illustrative products and methods of their use may drop the skin temp from the norm of 92-94° F. to about 65-82° F. degrees depending on the length of continuous time the products are utilized or about a 10-20° F. degree differential that is dependent on the starting temperature of the wrap.

The following describes temperatures of a cooling product and body according to an illustrative embodiment of the invention. The cooling product is refrigerated, which cools it to approximately 39° F. Within about 5 minutes of placement on the human body the product warms to approximately 61.4° F. and continues to raise in temperature at 15 minutes intervals to the following temperatures: 74.9° F., 68.0° F., 65.1° F. and 68.1° F. The product then levels off to approximately 68.1° F. and holds this temperature for about 2 hours of use. At room temperature the product is approximately 68.1° F. degrees and rises gradually over the next 60 minutes of utilization to approximately 71° F. and continues to hold this temperature as the evaporation process continues to dispel heat from the dermis and cool the various layers of skin and underlying tissues over the next 6-8 hours.

Various components of the solutions used to impregnate the substrate will now be described in more detail.

As noted above, illustrative gels are prepared using a polymer such as PVA, PVP or a combination of PVA and PVP. The PVP may be for example, BASF Luvitec® polymers. The surface tension of PVA and PVA/PVP blends is largely dependent on the degree of hydrolysis. Partially hydrolyzed grades yield solutions with the lowest surface tension and higher water sensitivity. Water sensitivity (dilution) can be further increased by adding sugars, glycerin, sorbitol (also known as “glucitol”), urea and salts, such as sodium nitrate and calcium chloride.

PVA is available with three major ranges of hydrolysis (99+, 96 to 98, and 86 to 89%). “Percent hydrolysis” is the percentage of the acetate groups in the starting material, polyvinyl acetate, that are hydrolyzed to alcohol groups. The desirable properties of fully hydrolyzed polyvinyl alcohol, i.e., with hydrolysis of around 98% or more, are decreased water sensitivity and increased tensile strength. Partially hydrolyzed polyvinyl alcohol, about 88% hydrolyzed, has increased water sensitivity and is a good dispersing agent.

The PVA is generally hydrolyzed in the range of about 96% to about less than 100%, with a particular embodiment of the invention including PVA that is about 99% hydrolyzed. Although the range and degree of aforementioned hydrolyzation are preferred for many applications, the invention includes super, fully, intermediate and tackified grades of PVA.

In an exemplary embodiment of the invention, fully hydrolyzed PVA is utilized, which allows for a higher degree of alcohol groups in the product, and a lesser amount of acetate groups from the parent compound. This enhances the cross-linking of the borate molecules to the alcohol when the gel is formed, which can lead to a strong, uniform, product.

Reaching a gelatin state can be better controlled when working with fully hydrolyzed PVA, since its solution has higher viscosity and the higher predominance of hydroxyl groups may contribute to cross-linking.

As noted above, a blend of PVA and PVP can be used. In a particular embodiment of the invention, the amount of PVA is greater than PVP based on weight. Other illustrative blends include, PVP greater than about 0 weight percent to about 2 weight percent, with a preferred range of about 0.25 weight percent to about 1.0 weight percent, and PVA about 4 weight percent to about 10 weight percent, with a preferred amount for some applications being 6 weight percent, wherein the weight percent is as measured with respect to the total aqueous solution, which will be described below. Other illustrative ranges include: about 4 to about 6 weight percent PVA and about 5 to about 15 weight percent PVP.

An illustrative range of K values for the PVP is about 88.0 to about 98.0; and about 27.0 to about 33.0. An illustrative range of PVP pH is about 3.0 to about 9.0. An illustrative PVP Glass Transition Temperature is about 180° C. An exemplary PVP Moisture Absorption at saturation (23° C., 75% relative humidity) value is about 40%.

Chemical and physical properties of PVA, PVP, and PVA/PVP blends may include nontoxicity, processability, good chemical resistance, wide range of crystallinity, good film formation capacity, complete biodegradability and high crystal modulus etc.

PVP, such as sold under the name Luvitec®, BASF for example, includes vinylpyrrolidone homopolymers and copolymers with different molecular weights. Homopolymers of vinylpyrrolidone, such as sold as Luvitec® K grade can be used. Copolymers of vinylpyrrolidone and vinylacetate monomers, such as Luvitec® VA grades, can also be used. These products can be in powder form or in aqueous solutions.

The degree of tack may be important when assembling a product as it helps keep parts together and in position. Durable adhesion over time may be also important. PVP brands such as Luvitec® provides both good initial tack and good adhesion over time.

Generally, PVP that has low toxicity, is non-irritating to the skin and eyes is suitable.

PVP, when cross-linked with water, becomes a hydrogel which is particularly adapted to skin adhesives applications. Once on the skin, it makes an impregnated substrate easy to remove with no harm.

The PVA or PVA/PVP blend can be plasticized by the addition of hydroscopic agents that retain water.

In general, a water-soluble plasticizer for the PVA, PVP, and PVA/PVP blends, which does not destroy the clarity of the gel is particularly suitable for many embodiments of the invention, although less clear or opaque gels are within the scope of the invention. Such plasticizers preferably soften the PVA, PVP, and PVA/PVP blends, provide a desirable stickiness, and assist in making the material easier to wash out of clothing.

Illustrative of such plasticizers are alkanes having from 2 to 5 carbon atoms and 2 to 3 hydroxyl groups such as glycerin; propylene glycol; ethylene glycol; and diethylene glycol; although ethylene glycol and diethylene glycol can have some toxic properties. Gel plasticizers may contribute to the durability, gloss, strength, and flexibility of the product. Glycerin (USP for example) is particularly useful in embodiments of the invention, as it is soluble in the polymer and decreases the Glass transition temperature (Tg) value. Other plasticizers can be used such as acetylated monoglycerides; alkyl citrates, sorbitol, Eastman Company DBP Plasticizer (dibutyl phthalate), and/or Ferro corporation Santicizer®.

The plasticizer transforms the PVA or PVA/PVP blend into a gel. The plasticizer is added to the solution in such amounts that the optimum degree of self-adherence of the material to itself is obtained. The PVA, PVP, or PVA/PVP blend will gel when it reacts with the inorganic compounds by a chemical cross-linking action. The gel that forms preferably has a solid-like consistency, is self-supporting, and is very flexible and compliant.

Triethylene glycol and glycerin can also be used as plasticizers. Glycerin is a good plasticizer because it lowers the dissolving temperature of the PVA, and raises the boiling point. Glycerin is utilized for numerous reasons including the fact that glycerin is virtually nontoxic to the environment and is essentially nontoxic by ingestion and harmless to the skin. It may also increase the shelf-life of the product.

In addition to using glycerin, or other plasticizers, PVA, PVP and PVA/PVP blends can be cross-linked using physical techniques such as heat treatment and radiation or chemical agents such as, boric acid. Chemical and mechanical properties of PVA, PVP, and PVA/PVP blends can be significantly changed by cross-linking. For example the increase in the degree of cross-linking can result in an increase in the melting point, decrease in the solubility, and increase in the tensile strength of the resulting polymer. In an illustrative embodiment of the invention, sodium borate is used to generate cross-linking, thereby improving the strength, flame retardant characteristics and flexibility of the finished product.

A defoaming agent may be introduced into one or both aqueous solutions used to impregnate a substrate with gel at a rate of less than 1% d/d, for example. Examples of suitable defoaming agents are Antifoam116 FG, Drewplus L474, Foamaster KB or Foamaster V.

The addition of sodium borate to a PVA/PVP blend or a PVA-treated substrate typically increases the fiber diameters and increases the viscosity of the solution. Other coagulating agents that can be utilized, for example at about 3 to 8 weight percent, are hydrofluoric acid, haxamethylene, hexaetylene diisocynate, glyoxal, glycols, such as dipropylene glycol, dibenzoate types, such as dipropylene glycol dibenzoate and diethylene glycol dibenzoate, phthalates, such as dibutyl phthalate, and liquid polyesters, such as triethylene glycol polyester of benzoic acid and phthalic acid, other humectants include calcium chloride, glycols, glycerine, ureas, sorbitol. Representative tackifiers include, gum rosin, ester gum, hydrocarbon resins, hydrogenated rosin, tall oil rosins, terpene resins, and others and others known in the water-based adhesion art.

Although there are a significant number of biocides that can kill microorganisms effectively and can provide very good preservation for polymer emulsions and other industrial products, only a limited number of these exhibit acceptably low toxicity to higher organisms, e.g., humans.

In an illustrative embodiment of the invention, Sodium hydroxymethylglycinate, which is a broad spectrum preservative, such as Suttocide® A 50% solution is used. The solution offers the benefits of a water soluble preservative as well as neutralizing agent for acids/acrylic polymers. Sodium hydroxymethylglycinate remains active at a pH as high as 12, and can be used in acidic conditions as low as pH 3.5. Sodium hydroxymethylglycinate is active against Gram-negative and Gram-positive bacteria, yeast and mold, even at low concentrations, providing cost-effective preservation.

Another illustrative example of antimicrobial agents is chlorhexidine. Chlorhexidine digluconate USP solution 5% is an aqueous solution of chlorhexidine digluconate 20% EP. Chlorhexidine 2-5% has a good bactericidal effect and is active against common Gram+ and Gram− bacteria and fungi. These, or any of the following antimicrobial agents can be added to the aqueous solutions, for example in an amount of about 2-5 weight percent chlorhexidine digluconate blended with 70% isopropyl alcohol or 60% ethyl alcohol, chlorhexidine digluconate and octenidine dihydrochloride blends, or other chlorhexidine blends, triclosan, PCMX, phenol (carbolic acid) compounds or a blend of 70% isopropyl alcohol or 60% ethyl alcohol or thymol (also known as 2-isopropyl-5-methylphenol), 4-Isopropyl-3-methylphenol or any concentration of isopropyl methylphenol or any concentration of a blend of thymol and carvacrol (cymophenol), Dow Chemical Kathon® LX 1400, Nuosept®, BASF's Protectol and Aseptrol for example along with similarly made antimicrobials, and Nanocide Antimicrobial finish. Additional examples of antimicrobial agents include silver sulfadiazine, silver nitrate 0.5% solution by percent weight, or Mafenide acetate (Sulfamylon) 5% solution by percent weight; Mupirocin (Bactroban); or Nystatin.

To enhance the cooling effect on skin, 1 to 7% by volume weight of oil of menthol, menthol crystals (Mentha arvensis Extraction-Crystallized quick frozen or similar) and/or menthol blends preferably 3% may be added to one or both aqueous solutions. Other cooling effect enhancements ingredients may be used, that chemically trigger cold-sensitive receptors in the skin to provide a cooling sensation.

Additionally, about 1 weight percent to about 5 weight percent of an anesthetic such as lidocaine (Xylocaine), lanacane, dibucaine, oxybuprocaine, pramoxine, proparacaine, proxymetacaine or other topical anesthetic can be added. Benzoate esters including benzocaine, benzocaine and menthol, tetracaine (also named amethocaine), and butamben picrate are other topical anesthetics that may be utilized. These agents may be added to one or both of the aqueous solutions. Topical anesthetics may aid itching, deaden nerve endings in the skin and can provide local pain relief.

The substrate may contain text, designs, and/or pictures, diagrams, and the like. (herein after referred to as “printing” wherein the term does not denote a particular process to impart “printing” on the substrate, but is merely used as shorthand) that will be visible even after the substrate is impregnated. The “printing” process and inks must be compatible with the impregnation process and associated materials. It is also possible for the text, designs, pictures, etc. to be a separate component that is adhered to the substrate either prior to or during the gel-impregnation process. The printing may be purely decorative or provide utility to the cooling product. For example the printing may provide directions for use. The printing may be imparted to the substrate by, for example, screen printing, roller printing, heat transfer printing, or other methods, and also can be woven or fused into the substrate. The printing must have fastness and be able to withstand the impregnation process, including the components of the solutions used.

The substrate may be pretreated with flame retardants such as polybrominated diphenyl ethers (PBDEs), fire poly EMC protectant and safe flame retardant compositions based on PVA and PVA oxidized by KMnO4 (polymer-organic char formers) or organic (non-salt) flame retardants.

Illustrative ultraviolet inhibitors containing ethyleneamine derivatives can be added.

Ethyleneamine derivatives can be added for their antistatic properties and quaternary ammonium compounds can be used with certain water-soluble neutral or alkaline salts, using organic agglomerating agents.

An illustrative method of forming the cooling product will now be described. A porous substrate is subjected, for example, by spraying, dipping or other form of immersion, to an aqueous solution having one or more polyvinyl compounds, such as PVA, PVP or a PVA/PVP blend until the substrate becomes substantially or completely impregnated with the solution. Illustrative weight percent ranges of the polyvinyl compounds in the solution are about 4 weight percent to about 6 weight percent; and about 4 weight percent to about 10 weight percent. The PVA/PVP blend may contain for example, PVP greater than about 0 weight percent to about 2 weight percent, with a preferred range of about 0.25 weight percent to about 1.0 weight percent, and PVA about 4 weight percent to about 10 weight percent, with a preferred amount for some applications being about 6 weight percent. Optionally, a range of about 2 to about 7 weight percent antimicrobial agent, such as chlorhexidine digluconate or Suttocide A 50% solution may be added. An additional illustrative range is about 3 to about 5 weight percent, preferably about 3 weight percent. The solution further comprises about 1 to 7 weight percent gel creating or enhancing component(s), such as glycerin, and the balance substantially water. Other illustrative ranges of gel creating or enhancing components include about 1 weight percent to about 5 weight percent; and about 3 weight percent to about 4 weight percent of the solution. Excess solution may be removed from the substrate by scraping with a blade or squeegee, pinch rolling, or a combination of techniques.

The wetted substrate is then subjected to another aqueous solution, again by spraying, dipping or other form of immersion. This second aqueous solution comprises about 3 to about 8 weight percent of an inorganic coagulating agent, such as sodium borate, to create cross-linking of the polymer, and optionally, about 2 to about 5 weight percent additional antimicrobial agent; and more than 0 to about 5 weight percent plasticizers, such as glycerin, and the balance essentially substantially water. Excess solution may be removed from the substrate, such as by scraping with a blade or squeegee, pinch rolling, or a combination of techniques. The impregnated substrate is then dried, for example by the use of air knives. The impregnated material will likely exhibit flame and heat resistance in excess of 3500° Fahrenheit.

Solution components and preparation according to an illustrative embodiment of the invention are as follows.

Aqueous Solution 1:

PVA 4-10 by weight percent, preferred is 6 by weight percent

PVP 0-2 by weight percent, preferred is 0.25-1.0 by weight percent

Antimicrobial agent 2-5 by weight percent, preferred is 3 by weight percent

Glycerin 1-7 by weight percent; preferred is 2-5 by weight percent;

The balance essentially water.

Aqueous Solution 2:

Sodium borate 3-8 by weight percent, preferred is 5 by weight percent

Antimicrobial agent 2-5 by weight percent, preferred is 2-3 by weight percent, more preferable 3 weight percent

Glycerin—0-7 by weight percent; preferred is 0-5 by weight percent; more preferable 1-2 weight percent

Other agents added would be at the percent weight indicated [e.g. menthol 1-7%, pain relievers, etc].

First Aqueous Solution

The first aqueous solution is heated to approximately 180° F. then allowed to cool to 160° F. or lower at which point, the antimicrobial agent, such as Suttocide A 50% solution, preferably 3 weight percent, is stirred into the aqueous solution.

Second Aqueous Solution

The sodium borate is added to warm water to dissolve the sodium borate, then the glycerin and antimicrobial agent are added.

In an exemplary embodiment of the invention, each aqueous solution is preferably prepared by mixing 60% of the water with the other ingredients making up the solution and heating the solution to approximately 180° F.-190° F. for the first aqueous solution and approximately 160° F.-170° F. for the second aqueous solution. The solution is continually agitated during the heating and mixing phase, preferably without a defoaming agent. Accordingly, the agitation is slow enough to avoid foaming of the solution. Generally it will take approximately 10-45 minutes to bring the solution to the aforementioned temperature. Once the desired temperature is reached, the solution components should be well blended, and sufficiently melted if necessary. The solution is then cooled by adding the remaining 40% of the water, at a temperature sufficient to cool the solution to less than 160° F. The antimicrobial agents are then added.

FIG. 1 depicts a cooling product manufacturing process and apparatus according to an illustrative embodiment of the invention. A substrate roll 50 contains untreated (non-impregnated) substrate material that is to be fed through the apparatus wherein it is impregnated with a polymer gel. The substrate is fed into a bath 52 of a first aqueous solution containing one or more polyvinyl compounds such as PVA and/or PVP or other similar substance, and additional ingredients as described herein. In this step, the substrate is preferably fed through the bath so it is fully immersed in the solution. Excess solution is then removed from the substrate. In this illustrative embodiment of the invention, the substrate is first fed through a pinch roller 54, which is formed from two different size rollers 56, 58. The substrate is then passed through a squeegee 60. Sprayers 62 spray the second aqueous solution onto the substrate to initiate or further cross-linking of the polymer(s). The substrate then follows a long path 64, which takes about 10-20 seconds to allow cross-linking to take place. Air knives 66 are then used to dry the impregnated substrate. Finally, the substrate is rewound onto a roller 68.

Some substrates, such as knits will tend to curl inward from the edges as they are stretched and/or impregnated with solution. Therefore, the apparatus contains decurlers to flatten the substrate. A first decurler 70 is located immediately before sprayer 62 to flatten the material so it uniformly receives the solution. A second decurler 72 is positioned downstream from sprayers 62, and immediately upstream from the take-up roller 68 so the material is flat as it is rolled onto roller 68.

The impregnation process time will depend on the rate of absorption of the selected substrate. The time can be reduced by mechanical compression of the substrate prior to impregnation so as to vacate air from the microcells. The gel preferably totally penetrates the substrate, but may only partially do so, forming a cooling product material.

The cross-linking of the PVA or PVA/PVP solutions with the coagulating solution may occur rapidly, usually within 1 to 60 seconds, depending on the properties of the substrate utilized. Ample time is required to assure effective migration of the coagulation solution throughout the impregnated substrate. The substrate may then be wiped of excessive gel using a blade, or other suitable device and allowed to dry to remove excess water. Drying time may be reduced by fully exposing the treated substrate to convective or radiative heat to evaporate the water, or by exposing the treated substrate to air knives to remove the excess moisture. In addition to or instead of the air knives, drying can be accomplished by utilizing a roller system that passes over air-drying fans, an infrared heating system, or other suitable drying mechanism. This may reduce drying time may be reduced to less than five minutes.

The impregnated substrate may then be formed into a variety of products, such as wraps, compresses, pads, patches, sleeves, vests, shirts, shorts, socks, hand coolers, foot coolers, head gear such as bands, caps or hats, t-shirts, shorts, pants, shirts, gloves, or other articles of clothing. The gel-impregnated substrates of the invention may also potentially be used as drug delivery systems. By combining the hydrogels with inorganic salts, they become conductive, and thus can also be used for electrosurgery.

The cooling product material can then be formed for example into, a cryotherapy wrap. An illustrative cryotherapy wrap thickness range is about 1/32 inch to about ¼ inch. The cryotherapy wrap will then be variously converted into finished product form. In the case of the cryotherapy wrap, the gel-impregnated substrate is cut for example, into sheets of approximately 1 to 8 inches in width by approximately 48 to 72 inches by length. In an exemplary embodiment of the invention, the wrap is rolled onto a core, having a diameter such as 0.5 to 1-½ inch. The wrap can be stored in a bag or other container that retains moisture, made for example of PVC or other plastic or plastic/metalized films. The wrap can re rehydrated by adding water to the storage container.

Although it is often preferable to create the cooling product material, and then form the material into the desired product, for some applications it is possible to form the desired product first from the substrate, and then impregnate with the gel and other components.

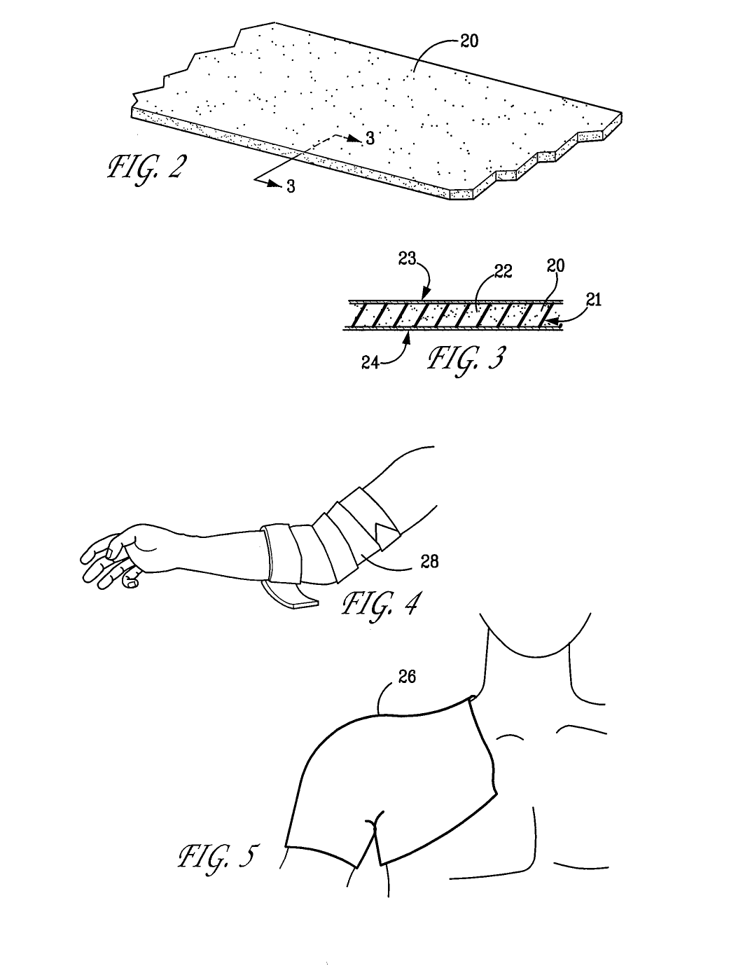

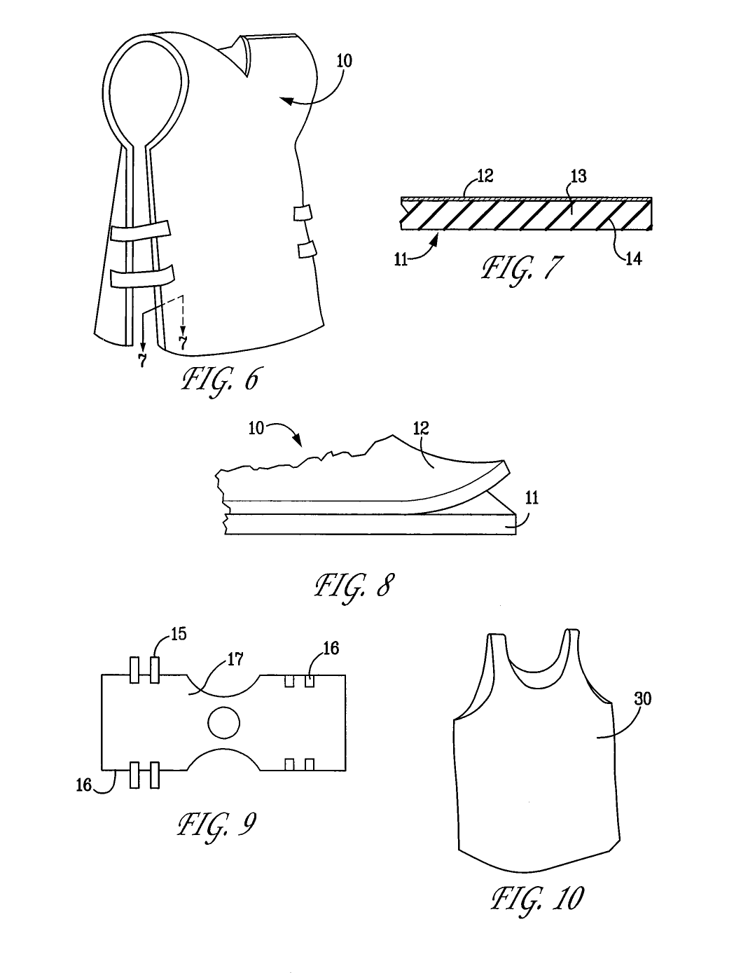

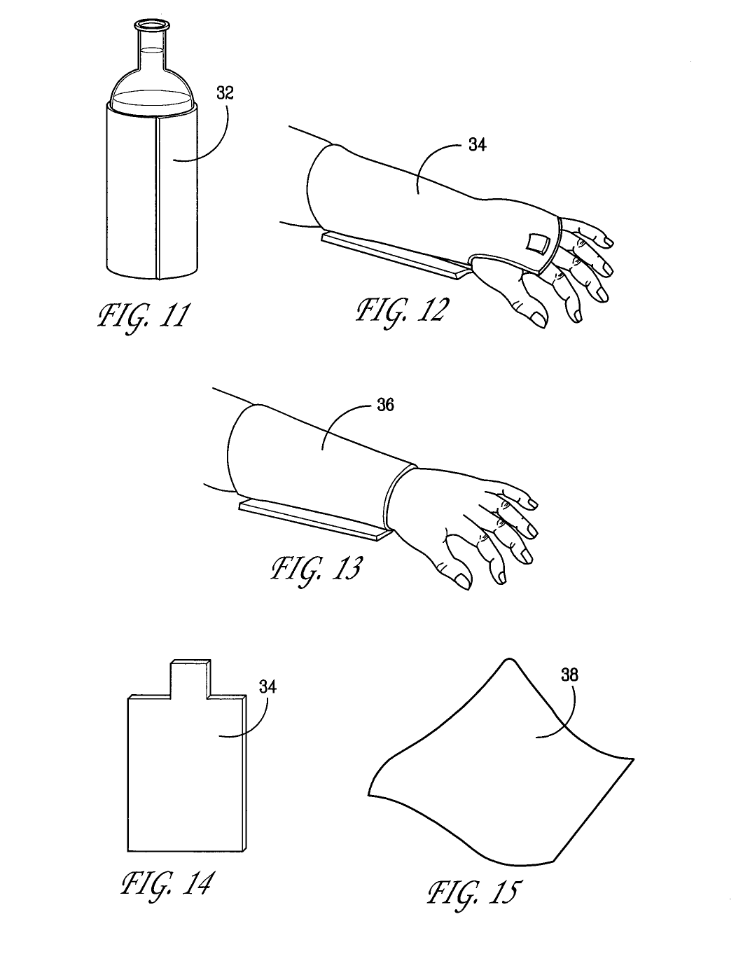

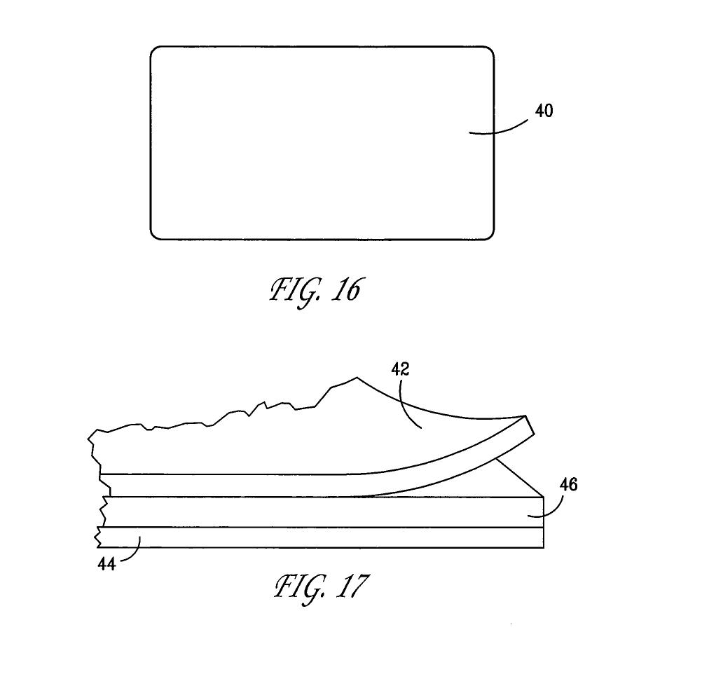

Referring to FIGS. 2 and 3, a cooling product material 20 comprises a carrier fabric 21 (substrate) that functions as a porous substrate for holding a water-based gel 22. This gel impregnates the carrier and provides cooling by drawing away body heat generally through a combination of thermal conductivity and evaporation. In an illustrative embodiment of the invention, the cooling product material surfaces 23, 24 are designed to be smooth and glossy while maintaining self-adhering properties that enable the wrap to adhere to itself, without adhering to the skin.

Examples of the substrate carrier 21 include, cotton over elastomeric polymer, such as Spandex® at about 1% to about 6% of a circular weft knit, of jersey construction. Additional illustrative ranges of elastomeric polymer include: about 2 to about 4 percent; and about 4 to about 6 percent.

There exist other forms of finished products that are encompassed within the scope of the invention including, but not limited to, patches and other treated products such as a bandage/wrap 25, which may include compression, as illustrated in FIG. 4, and a shoulder wrap 25, as illustrated in FIG. 5.

The finished products may be sterilized to allow for their use in surgical suites. Sterilization and decontamination can be accomplished by gamma irradiation. In an illustrative embodiment of the invention, the gamma sterilization process uses Cobalt 60 radiation to kill microorganisms. Gamma irradiation can penetrate packaging and product, and is suitable for use with most or all of the substrates used in embodiments of the invention.

A cryotherapy vest assembly according to an illustrative embodiment of the invention, offers a preferably flame and heat resistant, lightweight, and easy-to-use cryotherapy solution for keeping the body's core temperature at acceptable levels.

Referring to FIGS. 6-8, a cooling product in the form of a vest 10 comprises a impregnated substrate 11 that includes a porous carrier fabric 14, which holds a water-based gel 13. The impregnated substrate 11 provides cooling by drawing away body heat through a combination of thermal conductivity and evaporation. The vest may also include a thin fabric or plastic film 12 that is designed to enhance the evaporation of the water and thereby increase the cooling effect. As used herein, the term “vest” is used broadly and can include garments that in addition to covering chest and back areas, cover the shoulder or portions of the arm.

In an illustrative embodiment of the cryotherapy vest, the gel-impregnated substrate 11 is cut into sheets of approximately 18 to 36 inches by 36 inches. This size may be suitable for a cooling blanket 38 for example as shown in FIG. 15, to quickly reduce body temperature in a non-invasive manner. It can be formed from a single sheet of cooling product material 17, although it does not necessarily have to be. A thin fabric or plastic film 12, designed for moisture-wicking abilities may be applied to the carrier to enhance the cooling effect. The cut sheets are then cut into a vest pattern as shown in FIG. 9 that enables the user to slip the garment over his/her head and drape along the posterior and anterior sides of their body. Fasteners 15, 16 are attached to the sides of the garment, allowing the user to securely fasten the garment to their body. Fasteners 15, 16 may be for example, hook and loop materials that can be easily attached to one another, and repeatedly connected and disconnected. FIG. 10 depicts another garment 30 according to an illustrative embodiment of the invention. In this embodiment, the garment 30 is in the form of a tank top.

The final garment typically and preferably has a thickness of about 1/16 inch to ¼ inch to minimize the weight of the garment, while providing sufficient cooling and flame/heat resistance. The garment will typically and preferably be folded into a 12 inch by 16 inch standard sized PVC plastic bag, or other container that retains moisture. The PVC Plastic bag or other container will serve as a rehydration bag, allowing the user to reuse the product multiple times prior to disposal when the gel has completely dried out from repeated use.

The cooling product may also be in the form of a container wrap 32 such as shown in FIG. 11. Container wrap 32 can be used to chill and/or keep contained items cold, such as food and beverages or medicine during use or transport. In many cases, it eliminates or reduces the need to transport ice or ice water, and does not require an outside power source or cleanup due to condensation, melting, or spills. Illustrative embodiments of the invention may eliminate or reduce the cross contamination that occurs from unsanitary coolers and Cold Thermal Bags; the melting that occurs with ice and ice/water mixes in coolers or Cold Thermal Bags, and hands placed in melting water, the dripping on other cooler/Cold Thermal Bags contents from water when food products are removed from the cooler/Cold Thermal Bags.

Container wrap 32 can be wrapped around a bottle or other container, which will adhere to the container with little or no sticking, condensation or melting, and will provide cooling, or maintenance at a chilled temperature. Chilling wrap 32 can be rolled up and stored in a preferably airtight container, and re-used. Refrigeration enhances the cooling effects or submersion in cold or ice water, either while in a bag or other container or not. The wrap may also be submerged, generally for just a short time period (approximately 2-20 seconds—dependent on the overall size of the wrap—a larger wrap needs a longer immersion time) in cold or ice water then removing the excess water by hand squeezing or wiping off or refrigerated while wrapped around a container.

Container wrap 32 can be made with or without the ability to stretch. The ability to stretch can facilitate the cooling wrap conforming to the container. Examples would be a knit substrate consisting of cotton/spandex or similar, or a nonwoven material that is embedded with a gel that conforms to the sides of the vessel it is wrapped around. In an exemplary embodiment of the container wrap, the wrap has a size, between about ⅛ and about ½ inch thick, about 4 to about 36 inches wide, and about 10 to about 36 inches long. In a further embodiment, the wraps dimensions are between about ⅛ and about ½ inch thick, 7 to 10 inches wide, and about 9 to about 20 inches long. Due to the product's ability to adhere to itself, it can be combined in its present size and layered to provide a thicker, longer lasting product as needed. The non-impregnated substrate is preferably nonwoven polyester or polyester blend, weighing about 2 oz/yd² to about 4 oz/yd², preferably about 3 oz/yd², and having a thickness in the range of about 0.07 inches to about 0.10 inches.

The product can also be formed in various other sizes for other purposes, including but not limited to, burn pads, finger wraps, strips, face masks, leg wraps or other forms.

As shown in FIGS. 12 and 13, the invention includes body wraps that can be for example, used to cover a portion of the body, such as the wrist, arm or portion thereof. FIG. 12 shows a sleeve wrap 34 according to an illustrative embodiment of the invention that covers the wrist, a portion of the forearm and a portion of the hand. FIG. 13 shows another version of a sleeve wrap or forearm gauntlet 36 according to an illustrative embodiment of the invention that covers the wrist and a portion of the forearm. The illustrative embodiments of cooling products shown in FIGS. 12 and 13 are particularly useful for kitchen personnel, such as chefs. The wrap offers protection from burns and also aides in keeping the end user's dermis (skin) cool in a hot environment. By applying a cooling sleeve, wrap or gauntlet to the forearm at the start of a work shift, employees are protected against accidental scald, flame, hot grease and heat contact incidents. The wrap does not adhere to the skin, but does adhere to itself, and may be provided for example, in approximately 9-10 inch by 16 inch sheets of non-woven polyester fabric which has been treated with a polymer gel. In a further embodiment of the invention, an approximately nine inch width wrap, for example, provides good coverage for the forearm. A length of about 8 to about 18 inches would allow the wrap to be sufficiently wound around the forearm, and would provide a cooling effect for a reasonable amount of time.

The substrate for the sleeve or gauntlet is preferably a needlepunched, nonwoven polyester/rayon, such as SoftSorb HYG008. Ideally it will have a weight of 6 oz/yd², a thickness of 0.075-0.095 inches, a minimum tensile of 65 lbs in the machine direction (MD) and 100 lbs. in the cross direction (CMD), a mean percent elongation at 10 lbs of 40 (MD) and 25 (CMD), and a mean absorbency of 7.0 gm/gm. Other illustrative substrate ranges for sleeves and gauntlets are, thickness from about 0.05 to about 0.13 inches; weight about 3 lbs. oz/yd² to about 8 oz/yd².

Employees who have previously suffered a minor workplace burn or scald injury can protect the affected area with the sleeve wrap, as one of the wrap's characteristics is that the gel provides cooling of the skin, as well as a heat/flame bather.

The cooling product material can be formed into a pad 40, such as shown in FIGS. 16 and 17, for example in approximate 4 inch to 10 inch in width by 4 inches to 16 inch in length pieces. These can be applied to the body in a specific area, such as the lower back or shoulder. Ideally, the pads will have a release liner 42 on one side, such as made of polypropylene, and a fabric outer covering 44 pressed to the side of pad 40 worn away from the body to protect clothing that may be worn over the pad. Outer covering 44 can be the same material as the impregnated substrate 46, but will not be impregnated. The non-impregnated outer covering 44 should readily adhere to impregnated substrate 46 by use of a pinch roller for example. Preferably this is done while impregnated substrate 46 is sufficiently wet to facilitate adherence. Release liner 42 preferably has at least one textured side to help it adhere to impregnated substrate 46. The pad substrate will preferably be a needlepunched, nonwoven, polyester, such as Home Furnishing style VYL009, which has a weight of about 1.50 oz·yd², and a thickness of about 0.017-0.033 inches. Other illustrative ranges of weight for pads include about 1 oz/yd² to about 3 oz/yd². Other illustrative ranges of thickness for pads includes about 0.010 to about 0.050.

The invention may be embodied in a variety of ways, for example, a product such as a cooling pad, a method of making an impregnated substrate for cooling applications, a method of making a cooling product, a method of cooling a body, a method of cryotherapy, a method of treating burns, a method of reducing swelling, and a method of treating injuries, comprising applying any product described herein to the body to provide a cooling effect. Embodiments of the cryotherapy method include administering the therapy during the acute inflammatory phase—the first 24 to 48 hours after injury, and at a temperature above about 59° F. (15° C.) to avoid or diminish the possibility or extent of vasodilatation and cell damage or cell death.

Various embodiments of the invention have been described, each having a different combination of elements. The invention is not limited to the specific embodiments disclosed, and may include different combinations of the elements disclosed and may include aspects of the prior art.

While the invention has been described by illustrative embodiments, additional advantages and modifications will occur to those skilled in the art. Therefore, the invention in its broader aspects is not limited to specific details shown and described herein. Modifications, for example, to the ratios of material components and product configurations and types, may be made to at least some extent without departing from the spirit and scope of the invention. Accordingly, it is intended that the invention not be limited to the specific illustrative embodiments, but be interpreted within the full spirit and scope of the appended claims and their equivalents.

What is claimed is:

1. A method of forming a cooling product comprising:
subjecting a porous substrate to a first aqueous solution comprised of about 2 to about 10 weight percent of one or more polyvinyl compounds, about 2 to about 5 weight percent antimicrobial agent, about 1 to 7 weight percent gel creating or enhancing component(s), and the balance substantially water; and
subjecting the wetted substrate to a second aqueous solution comprised of about 3 to 8 weight percent of an inorganic coagulating agent, about 2 to about 5 weight percent antimicrobial agent; and about more than 0 to about 5 weight percent plasticizers and the balance substantially water.

2. The method of claim 1 wherein the polyvinyl compound is PVA.

3. The method of claim 1 wherein the polyvinyl compound is a PVA/PVP blend.

4. The method of claim 3 wherein the PVP is in the range of greater than about 0 weight percent to about 2 weight percent, and the PVA is about 4 weight percent to about 10 weight percent of the first aqueous solution.

5. The cooling product of claim 4 wherein the PVP is in the range of about 0.25 weight percent to about 1.0 weight percent.

6. The method of claim 1 wherein the first aqueous solution comprises about 2 weight percent to about 7 weight percent antimicrobial agent.

7. The method of claim 6 wherein the antimicrobial agent is sodium hydroxymethylglycinate.

8. The method of claim 1 wherein the plasticizer is glycerin.

9. The method of claim 1 wherein the inorganic coagulating agent is sodium borate.

10. The method of claim 1 wherein the PVA is about 99% hydrolyzed.

11. The method of claim 1 wherein the total concentration of one or more polyvinyl compounds is about 4 weight percent to about 6 weight percent.

12. The method of claim 1 wherein the substrate is a knit, elastomeric polymer/cotton blend.

13. The method of claim 1 further comprising forming the cooling product into a compression wrap.

14. The method of claim 1 further comprising forming the cooling product into a vest.

15. The method of claim 1 further comprising forming the cooling product into a container wrap.

16. The method of claim 1 further comprising forming the cooling product into a pad, body wrap or blanket.

17. The method of claim 1 further comprising adding inorganic salts.

18. A method of cooling a body or object comprising:
applying the product of claim 1 to the body or object.
